(19) United States
(12) Patent Application Publication
(10) Pub. No.: US 2022/0312709 A1
(43) Pub. Date: Oct. 6, 2022

(54) HIGH-EFFICIENCY ZEA MAYS L. BREEDING METHOD BASED ON INDIVIDUAL PLANT EVALUATION AND GENOME-WIDE SELECTION (GWS)

(71) Applicants: JIANGSU ACADEMY OF AGRICULTURAL SCIENCES, Nanjing (CN); INSTITUTE OF TOBACCO, ANHUI ACADEMY OF AGRICULTURAL SCIENCES, Hefei (CN)

(72) Inventors: Tifu ZHANG, Nanjing (CN); Yiliang QIAN, Nanjing (CN); Han ZHAO, Nanjing (CN); Long RUAN, Nanjing (CN)

(73) Assignees: JIANGSU ACADEMY OF AGRICULTURAL SCIENCES, Nanjing (CN); INSTITUTE OF TOBACCO, ANHUI ACADEMY OF AGRICULTURAL SCIENCES, Hefei (CN)

(21) Appl. No.: 17/846,044

(22) Filed: Jun. 22, 2022

Related U.S. Application Data
(63) Continuation of application No. PCT/CN2021/142034, filed on Dec. 28, 2021.

(30) Foreign Application Priority Data
Feb. 5, 2021 (CN) 202110164476.9
Dec. 6, 2021 (CN) 202111477328.9

(51) Int. Cl. A01H 1/04

(52) U.S. Cl. CPC A01H 1/04

(57) ABSTRACT

A high-efficiency Zea mays L. breeding method based on individual plant evaluation and genome-wide selection (GWS) includes: S1. in a first cropping season, pollinating a Zea mays L. female parent with multiple male parents; S2. in a second cropping season, subjecting hybrid seeds to single-seed sowing, conducting individual plant selection, and evaluating target traits; S3. identifying a parent of a selected cross combination; S4. subjecting a target trait of a cross combination to genome-wide prediction; S5. selecting an excellent cross combination according to a predicted target trait; and S6. subjecting the selected excellent cross combination directly to variety registration or to further evaluation. The high-efficiency Zea mays L. breeding method provided by the present disclosure greatly reduces a quantity of cross combination seeds obtained in the first cropping season and a planting scale of the cross combinations in the second cropping season, and effectively reduces the breeding cost.

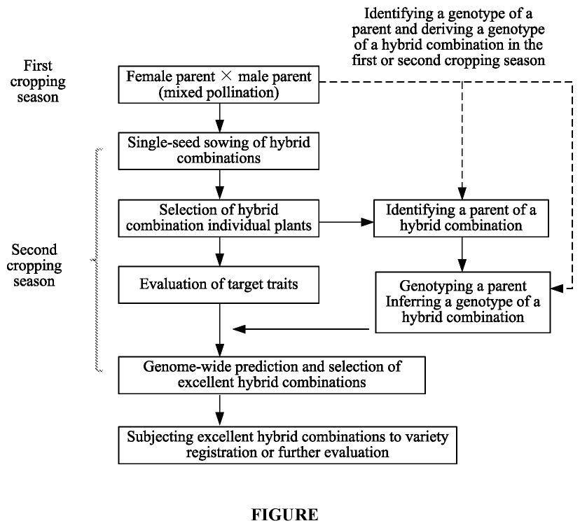

### CROSS REFERENCE TO THE RELATED APPLICATIONS

This application is a continuation application of International Application No. PCT/CN2021/142034, filed on Dec. 28, 2021, which is based upon and claims priority to Chinese Patent Applications No. 202111477328.9 filed on Dec. 6, 2021 and No. 202110164476.9, filed on Feb. 5, 2021, the entire contents of which are incorporated herein by reference.

### TECHNICAL FIELD

The present disclosure relates to a high-efficiency Zea mays L. breeding method based on individual plant evaluation and genome-wide selection (GWS).

### BACKGROUND

Zea mays L. is the main field crop worldwide, and the breeding of new high-yield Zea mays L. varieties is one of the important ways to increase a yield. According to the conventional Zea mays L. breeding method, a Zea mays L. breeding unit usually needs to make thousands or even tens of thousands of cross combinations every year and evaluate these cross combinations through multiple-row plots at multiple locations in the next cropping season to screen out a few excellent cross combinations for variety registration. In this process, a large number of useless cross combinations need to be produced and subjected to multi-location evaluation, which consumes a lot of manpower, material resources, and financial resources, and is not conducive to the control of a breeding cost by a breeding unit. In addition, the cross combinations obtained in breeding practice only account for a small part of a theoretical quantity, and thus excellent cross combinations may not be made and screened out, resulting in a waste of excellent genetic resources.

### SUMMARY

In order to solve the problem in the prior art that excellent cross combinations may not be made and screened out because the cross combinations obtained in Zea mays L. breeding only account for a small part of a theoretical quantity, which results in a waste of excellent genetic resources, the present disclosure provides a high-efficiency Zea mays L. breeding method based on individual plant evaluation and GWS.

To solve the above technical problem, the present disclosure provides the following technical solution.

A high-efficiency Zea mays L. breeding method based on individual plant evaluation and GWS is provided, including the following steps:

S1. in a first cropping season, pollinating a Zea mays L. female parent with multiple male parents;

S2. in a second cropping season, subjecting hybrid seeds to single-seed sowing, conducting individual plant selection, and evaluating target traits, where when the hybrid seeds are subjected to single-seed sowing, a female parent corresponding to a hybrid seed subjected to single-seed sowing is recorded; and the sowing density, soil conditions, field management measures, and other factors affecting the growth of individual plants should be consistent;

S3. identifying a parent of a selected cross combination, which specifically includes the following steps:

S31. screening genomic DNAs (gDNAs) of all male and female parents in the first cropping season to obtain molecular markers used to identify parental genotypes;

S32. subjecting gDNA templates of all male and female parents in the first cropping season and the hybrid seeds to polymerase chain reaction (PCR) amplification with the molecular markers, and recording genotypes;

S33. deriving all possible cross combination genotypes according to genotypes of all male and female parents in the first cropping season; and

S34. comparing genotypes of the hybrid seeds with all cross combination genotypes derived, and recording a matching rate of identical genotype loci, where if a selected cross combination and a derived cross combination have the highest matching rate of identical genotype loci, a parent of the derived cross combination is a parent of the selected cross combination;

S4. subjecting a target trait of a cross combination to genome-wide prediction, where

S4 specifically includes the following steps:

S41. genotyping a parent of a cross combination identified in S3, and inferring a genotype of the corresponding cross combination according to a genotype of the parent;

S42. using target trait averages and genotypes of cross combinations evaluated in S2 to fit a genome-wide prediction model; and

S43. predicting target traits of all possible cross combinations according to the fitted genome-wide prediction model;

S5. selecting an excellent cross combination according to a predicted target trait; and

S6. subjecting the selected excellent cross combination directly to variety registration or to further evaluation.

The identification of a genotype of a parent and the derivation of a genotype of a cross combination are not limited to the second cropping season and can be completed in the first cropping season.

In theory, as long as the genotype information of Zea mays L. inbred lines and the phenotypic values (such as yield, quality, and resistance) of some cross combinations are acquired, genomic estimated breeding values (GEBVs) of corresponding phenotypes of all remaining cross combinations can be predicted, and according to prediction results, excellent combinations can be selected for variety registration, which avoids the evaluation of a large number of useless combinations. Therefore, GWS is conducive to the development of the high-efficiency Zea mays L. breeding technology, which can effectively reduce the breeding cost and has an important application value for the Zea mays L. breeding of breeding companies at a controlled cost.

The present disclosure has the following beneficial effects:

The present disclosure proposes for the first time the combination of individual plant trait evaluation and GWS for Zea mays L. cross combinations, which can efficiently select a target cross combination and reduce the breeding cost.

Because it is not necessary to conduct multiple-row plot evaluation on the cross combinations, a quantity of cross combination seeds obtained in the first cropping season and a planting scale of the cross combinations in the second cropping season are greatly reduced and the breeding cost is effectively reduced.

The established GWS can predict cross combinations that are not produced to screen excellent combinations, which can reduce the waste of excellent genetic resources and improve the selection efficiency of breeding practice.

### BRIEF DESCRIPTION OF THE DRAWINGS

The accompanying drawing is used to further illustrate the present disclosure and constitutes a part of the specification. The accompanying drawing, together with the example of the present disclosure, is provided to explain the present disclosure, but does not constitute a limitation to the present disclosure.

FIGURE is a flow chart of the high-efficiency Zea mays L. breeding method based on individual plant evaluation and GWS.

### DETAILED DESCRIPTION OF THE EMBODIMENTS

The preferred example of the present disclosure is described below with reference to the accompanying drawing. It should be understood that the preferred example described herein is only used to illustrate and explain the present disclosure, rather than to limit the present disclosure.

### EXAMPLE

A high-efficiency Zea mays L. breeding method based on individual plant evaluation and GWS was provided, including the following steps:

(1) Production of Cross Combinations

In the winter of 2018, 10 female parent inbred lines and 6 male parent inbred lines were selected and planted in Hainan. The female parent inbred lines were planted with two rows on each ridge, and the male parent inbred lines were mixed-planted with two rows on each ridge. A mixed male parent inbred line row was planted following every 2 adjacent female parent inbred line rows. The tassel was removed from a female parent material before silking, and then the female parent material was open-pollinated. Seeds of a same female parent inbred line were harvested together and sown in the next season.

(2) Single-Seed Sowing of Cross Combinations

Seeds of the cross combinations harvested in the last season were sown in Fuyang, Anhui in the summer of 2019. A test field was flat and had uniform soil fertility. 300 hybrid seeds of a same female parent were randomly selected and directly sown independently at a same density (25 seeds/row). 8 commercial hybrid seeds (parents thereof were among the 16 parents) were sown as controls. Field management during the growth of Zea mays L. were the same.

(3) Trait Evaluation of Individual Hybrid Plant

At a maturity stage of Zea mays L., breeders selected 134 individual hybrid plants with excellent comprehensive traits based on experience and harvested. The amount of hybrid derived from the same female parent ranges from 1 to 37. Single ear threshing was conducted, and resulting grains on each ear were oven-dried and weighed to obtain a grain weight per ear.

(4) Identification of Parents of Cross Combinations

gDNA was extracted from leaves of 134 selected cross combination plants, 8 commercial hybrid controls, and 16 parents. All cross combinations and parents were subjected to PCR amplification using 33 molecular markers screened from the parents, and amplification products were analyzed by agarose gel electrophoresis. For each molecular marker, a short band was recorded as A, a long band was recorded as B, a double-band was recorded as H, and a missing was recorded as N. A genotype of a cross combination was derived according to the following criteria: when either parent of a cross combination is N, a genotype of the cross combination is recorded as N; when genotypes of both parents of a cross combination are both A or B, a genotype of the cross combination is A or B; and when genotypes of both parents of a cross combination are respectively A and B, a genotype of the cross combination is H. Theoretically, a maximum of 60 cross combinations can be obtained from the 10 female parent inbred lines and the 6 male parent inbred lines. The genotypes of 33 marker loci in 142 cross combinations were compared with the derived genotypes of 60 cross combinations, and a number of identical genotype loci and a number of non-missing loci were recorded. A matching rate of identical genotype loci was calculated as follows: matching rate of identical genotype loci (%)=number of identical genotype loci/number of non-missing loci×100. When a matching rate of identical genotype loci is the highest, a parent of a derived genotype cross combination was considered as a parent of a corresponding selected cross combination (Table 1). 8 commercial hybrids as controls were each matched to a correct derived cross combination, which verified the accuracy of the method for identifying a parent. According to analysis, the selected 134 cross combinations were actually 39 different cross combinations, and 1 to 14 plants were selected for each cross combination (Table 1).

(5) Genome-Wide Prediction of Target Traits

The 16 parent inbred lines were genotyped using the illumina MaizeSNP50 BeadChip. Missing and heterozygous single nucleotide polymorphism (SNP) loci and SNP loci with minor allele frequency (MAF) of less than 0.05 were filtered out, and then the remaining 31,260 SNP loci were used to derive genotypes of the 60 cross combinations. Derivation criteria were as follows: when genotypes of both parents of a cross combination are both A or B, a genotype of the cross combination is A or B; and when genotypes of both parents of a cross combination are respectively A and B, a genotype of the cross combination is H. The derived genotypes of 39 cross combinations and an average grain weight per ear of the cross combinations were fitted by the R-rrBLUP v4.6 package to obtain a molecular marker effect. According to the rrBLUP model, GEBVs of grain weight per ear of the 60 cross combinations could be estimated with the deduced genotypes and 31,260 SNP molecular marker effects of the 60 cross combinations to realize the genome-wide prediction of target traits.

(6) Selection of Excellent Cross Combinations

The GEBVs of grain weight per ear of the 60 cross combinations were ranked from largest to smallest (Table 2), and the top 10% of the cross combinations were selected for subsequent breeding (the selection criteria were determined by breeders). The top 10% of the cross combinations include two commercial Zea mays L. varieties, of which Xianyu 335 is currently a Zea mays L. variety with the largest planting area in China. The results indicate the high efficiency of GWS. According to the results, breeders can determine whether the selected cross combinations are subjected directly to variety registration or to further evaluation.

TABLE 1
Parental identification of cross combinations

| | | | | | Highest |
|---|---|---|---|---|---|
| | | | | | matching rate |
| | Corresponding | | Number of | Number of | of identical |
| Individual | commercial hybrid | | identical | non-missing | genotype |
| plant No. | or female parent | Matched derived cross combination | loci | loci | loci (%) |
| DFDM19001-CK | Denghai 605 | 605 female parent × 605 male parent | 24 | 33 | 72.7 |
| DFDM19002-12 | 605 female parent | 605 female parent × B6232 | 27 | 33 | 81.8 |
| DFDM19002-17 | 605 female parent | 605 female parent × B6232 | 29 | 33 | 87.9 |
| DFDM19002-18 | 605 female parent | 605 female parent × B6232 | 30 | 33 | 90.9 |
| DFDM19002-19 | 605 female parent | 605 female parent × B6232 | 31 | 33 | 93.9 |
| DFDM19002-25 | 605 female parent | 605 female parent × B6232 | 23 | 31 | 74.2 |
| DFDM19002-27 | 605 female parent | 605 female parent × B6232 | 31 | 33 | 93.9 |
| DFDM19002-30 | 605 female parent | 605 female parent × B6232 | 28 | 33 | 84.8 |
| DFDM19002-31 | 605 female parent | 605 female parent × B6232 | 27 | 33 | 81.8 |
| DFDM19002-35 | 605 female parent | 605 female parent × B6232 | 26 | 33 | 78.8 |
| DFDM19002-26 | 605 female parent | 605 female parent × B6275 | 25 | 33 | 75.8 |
| DFDM19002-1 | 605 female parent | 605 female parent × PH4CV | 16 | 32 | 50.0 |
| DFDM19002-16 | 605 female parent | 605 female parent × PH4CV | 20 | 31 | 64.5 |
| DFDM19002-20 | 605 female parent | 605 female parent × PH4CV | 23 | 31 | 74.2 |
| DFDM19002-21 | 605 female parent | 605 female parent × 618 male parent | 24 | 33 | 72.7 |
| DFDM19003-CK | Denghai 618 | 618 female parent × 618 male parent | 31 | 33 | 93.9 |
| DFDM19004-1 | 618 female parent | 618 female parent × B6211 | 27 | 33 | 81.8 |
| DFDM19004-4 | 618 female parent | 618 female parent × B6211 | 28 | 33 | 84.8 |
| DFDM19004-6 | 618 female parent | 618 female parent × B6211 | 32 | 33 | 97.0 |
| DFDM19004-7 | 618 female parent | 618 female parent × B6211 | 25 | 33 | 75.8 |
| DFDM19004-10 | 618 female parent | 618 female parent × B6211 | 29 | 33 | 87.9 |
| DFDM19004-15 | 618 female parent | 618 female parent × B6211 | 30 | 33 | 90.9 |
| DFDM19004-16 | 618 female parent | 618 female parent × B6211 | 32 | 33 | 97.0 |
| DFDM19004-12 | 618 female parent | 618 female parent × B6232 | 28 | 33 | 84.8 |
| DFDM19004-17 | 618 female parent | 618 female parent × B6232 | 26 | 33 | 78.8 |
| DFDM19004-22 | 618 female parent | 618 female parent × B6232 | 27 | 33 | 81.8 |
| DFDM19004-25 | 618 female parent | 618 female parent × B6232 | 27 | 33 | 81.8 |
| DFDM19004-5 | 618 female parent | 618 female parent × B6275 | 23 | 31 | 74.2 |
| DFDM19004-20 | 618 female parent | 618 female parent × PH4CV | 29 | 32 | 90.6 |
| DFDM19004-26 | 618 female parent | 618 female parent × PH4CV | 29 | 33 | 87.9 |
| DFDM19004-28 | 618 female parent | 618 female parent × PH4CV | 22 | 32 | 68.8 |
| DFDM19004-31 | 618 female parent | 618 female parent × PH4CV | 30 | 33 | 90.9 |
| DFDM19004-33 | 618 female parent | 618 female parent × PH4CV | 31 | 33 | 93.9 |
| DFDM19004-13 | 618 female parent | 618 female parent × 605 male parent | 19 | 33 | 57.6 |
| DFDM19004-2 | 618 female parent | 618 female parent × 618 male parent | 31 | 33 | 93.9 |
| DFDM19004-9 | 618 female parent | 618 female parent × 618 male parent | 24 | 33 | 72.7 |
| DFDM19006-1 | DK517 female parent | DK517 female parent × B6211 | 21 | 33 | 63.6 |
| DFDM19006-2 | DK517 female parent | DK517 female parent × B6211 | 31 | 33 | 93.9 |
| DFDM19006-3 | DK517 female parent | DK517 female parent × B6211 | 24 | 33 | 72.7 |
| DFDM19006-4 | DK517 female parent | DK517 female parent × B6211 | 31 | 33 | 93.9 |
| DFDM19006-5 | DK517 female parent | DK517 female parent × B6211 | 29 | 33 | 87.9 |
| DFDM19006-7 | DK517 female parent | DK517 female parent × B6211 | 26 | 33 | 78.8 |
| DFDM19006-11 | DK517 female parent | DK517 female parent × B6211 | 30 | 33 | 90.9 |
| DFDM19006-15 | DK517 female parent | DK517 female parent × B6211 | 26 | 33 | 78.8 |
| DFDM19006-17 | DK517 female parent | DK517 female parent × B6211 | 29 | 33 | 87.9 |
| DFDM19006-21 | DK517 female parent | DK517 female parent × B6211 | 27 | 33 | 81.8 |
| DFDM19006-8 | DK517 female parent | DK517 female parent × B6232 | 26 | 33 | 78.8 |
| DFDM19006-13 | DK517 female parent | DK517 female parent × B6232 | 30 | 33 | 90.9 |
| DFDM19006-23 | DK517 female parent | DK517 female parent × B6232 | 26 | 33 | 78.8 |
| DFDM19006-44 | DK517 female parent | DK517 female parent × B6232 | 28 | 33 | 84.8 |
| DFDM19006-10 | DK517 female parent | DK517 female parent × B6275 | 22 | 32 | 68.8 |
| DFDM19006-24 | DK517 female parent | DK517 female parent × B6275 | 32 | 33 | 97.0 |
| DFDM19006-25 | DK517 female parent | DK517 female parent × B6275 | 30 | 33 | 90.9 |
| DFDM19006-28 | DK517 female parent | DK517 female parent × B6275 | 20 | 30 | 66.7 |
| DFDM19006-31 | DK517 female parent | DK517 female parent × B6275 | 26 | 33 | 78.8 |
| DFDM19006-43 | DK517 female parent | DK517 female parent × B6275 | 28 | 33 | 84.8 |
| DFDM19006-14 | DK517 female parent | DK517 female parent × PH4CV | 29 | 33 | 87.9 |
| DFDM19006-16 | DK517 female parent | DK517 female parent × PH4CV | 29 | 33 | 87.9 |
| DFDM19006-18 | DK517 female parent | DK517 female parent × PH4CV | 29 | 33 | 87.9 |
| DFDM19006-20 | DK517 female parent | DK517 female parent × PH4CV | 30 | 33 | 90.9 |
| DFDM19006-22 | DK517 female parent | DK517 female parent × PH4CV | 28 | 33 | 84.8 |
| DFDM19006-29 | DK517 female parent | DK517 female parent × PH4CV | 29 | 33 | 87.9 |
| DFDM19006-34 | DK517 female parent | DK517 female parent × PH4CV | 27 | 33 | 81.8 |
| DFDM19006-35 | DK517 female parent | DK517 female parent × PH4CV | 28 | 33 | 84.8 |
| DFDM19006-36 | DK517 female parent | DK517 female parent × PH4CV | 30 | 33 | 90.9 |
| DFDM19006-38 | DK517 female parent | DK517 female parent × PH4CV | 29 | 33 | 87.9 |
| DFDM19006-40 | DK517 female parent | DK517 female parent × PH4CV | 28 | 33 | 84.8 |
| DFDM19006-41 | DK517 female parent | DK517 female parent × PH4CV | 25 | 33 | 75.8 |
| DFDM19006-42 | DK517 female parent | DK517 female parent × PH4CV | 27 | 33 | 81.8 |
| DFDM19006-46 | DK517 female parent | DK517 female parent × PH4CV | 29 | 33 | 87.9 |
| DFDM19006-26 | DK517 female parent | DK517 female parent × 618 male | 26 | 33 | 78.8 |
| | | parent | | | |
| DFDM19006-27 | DK517 female parent | DK517 female parent × 618 male | 22 | 33 | 66.7 |
| | | parent | | | |
| DFDM19006-45 | DK517 female parent | DK517 female parent × 618 male | 29 | 33 | 87.9 |
| | | parent | | | |
| DFDM19007-CK | Yayu No. 2 | A7016 × B6275 | 21 | 32 | 65.6 |
| DFDM19008-1 | A7016 | A7016 × B6211 | 30 | 33 | 90.9 |
| DFDM19008-2 | A7016 | A7016 × B6211 | 29 | 33 | 87.9 |
| DFDM19008-7 | A7016 | A7016 × B6211 | 27 | 33 | 81.8 |
| DFDM19008-13 | A7016 | A7016 × B6211 | 27 | 33 | 81.8 |
| DFDM19008-15 | A7016 | A7016 × B6211 | 28 | 33 | 84.8 |
| DFDM19008-40 | A7016 | A7016 × B6211 | 28 | 33 | 84.8 |
| DFDM19008-3 | A7016 | A7016 × B6232 | 24 | 33 | 72.7 |
| DFDM19008-18 | A7016 | A7016 × B6275 | 25 | 33 | 75.8 |
| DFDM19008-36 | A7016 | A7016 × B6275 | 26 | 33 | 78.8 |
| DFDM19008-20 | A7016 | A7016 × PH4CV | 29 | 33 | 87.9 |
| DFDM19008-23 | A7016 | A7016 × PH4CV | 26 | 33 | 78.8 |
| DFDM19008-25 | A7016 | A7016 × PH4CV | 28 | 33 | 84.8 |
| DFDM19008-26 | A7016 | A7016 × PH4CV | 27 | 33 | 81.8 |
| DFDM19008-28 | A7016 | A7016 × PH4CV | 28 | 33 | 84.8 |
| DFDM19008-32 | A7016 | A7016 × PH4CV | 27 | 33 | 81.8 |
| DFDM19008-12 | A7016 | A7016 × 618 male parent | 23 | 32 | 71.9 |
| DFDM19008-29 | A7016 | A7016 × 618 male parent | 29 | 33 | 87.9 |
| DFDM19008-34 | A7016 | A7016 × 618 male parent | 31 | 32 | 96.9 |
| DFDM19008-38 | A7016 | A7016 × 618 male parent | 29 | 33 | 87.9 |
| DFDM19008-6 | A7016 | A7016 × B6232 | 18 | 33 | 54.5 |
| DFDM19009-CK | Luyan 188 | A8001 × B6211 | 29 | 33 | 87.9 |
| DFDM19010-4 | A8001 | A8001 × B6211 | 26 | 33 | 78.8 |
| DFDM19010-5 | A8001 | A8001 × B6211 | 29 | 33 | 87.9 |
| DFDM19010-6 | A8001 | A8001 × B6211 | 29 | 33 | 87.9 |
| DFDM19010-8 | A8001 | A8001 × B6211 | 28 | 33 | 84.8 |
| DFDM19010-12 | A8001 | A8001 × B6211 | 28 | 33 | 84.8 |
| DFDM19010-15 | A8001 | A8001 × B6211 | 26 | 33 | 78.8 |
| DFDM19010-24 | A8001 | A8001 × B6275 | 20 | 30 | 66.7 |
| DFDM19010-32 | A8001 | A8001 × B6275 | 30 | 33 | 90.9 |
| DFDM19010-52 | A8001 | A8001 × B6275 | 30 | 33 | 90.9 |
| DFDM19010-18R | A8001 | A8001 × PH4CV | 30 | 33 | 90.9 |
| DFDM19010-22R | A8001 | A8001 × PH4CV | 30 | 33 | 90.9 |
| DFDM19010-23R | A8001 | A8001 × PH4CV | 30 | 33 | 90.9 |
| DFDM19010-25R | A8001 | A8001 × PH4CV | 30 | 33 | 90.9 |
| DFDM19010-26R | A8001 | A8001 × PH4CV | 30 | 33 | 90.9 |
| DFDM19010-28R | A8001 | A8001 × PH4CV | 30 | 33 | 90.9 |
| DFDM19010-37 | A8001 | A8001 × 605 male parent | 15 | 33 | 45.5 |
| DFDM19010-13R | A8001 | A8001 × 605 male parent | 28 | 33 | 84.8 |
| DFDM19011-CK | Lvyu 178 | A8111 × B6211 | 29 | 33 | 87.9 |
| DFDM19012-2 | A8111 | A8111 × B6211 | 31 | 33 | 93.9 |
| DFDM19012-13 | A8111 | A8111 × B6211 | 21 | 33 | 63.6 |
| DFDM19012-14 | A8111 | A8111 × B6211 | 30 | 33 | 90.9 |
| DFDM19012-15 | A8111 | A8111 × B6211 | 31 | 33 | 93.9 |
| DFDM19012-9 | A8111 | A8111 × B6275 | 22 | 33 | 66.7 |
| DFDM19012-34 | A8111 | A8111 × PH4CV | 29 | 33 | 87.9 |
| DFDM19012-11 | A8111 | A8111 × 618 male parent | 19 | 28 | 67.9 |
| DFDM19013-2 | YD9953 female parent | YD9953 female parent × B6211 | 24 | 33 | 72.7 |
| DFDM19013-3 | YD9953 female parent | YD9953 female parent × B6211 | 25 | 33 | 75.8 |
| DFDM19013-8 | YD9953 female parent | YD9953 female parent × B6211 | 25 | 33 | 75.8 |
| DFDM19013-11 | YD9953 female parent | YD9953 female parent × B6275 | 17 | 24 | 70.8 |
| DFDM19013-1 | YD9953 female parent | YD9953 female parent × PH4CV | 29 | 33 | 87.9 |
| DFDM19014-CK | Yufeng 303 | 303 female parent × PH4CV | 30 | 33 | 90.9 |
| DFDM19015-7 | 303 female parent | 303 female parent × B6275 | 32 | 33 | 97.0 |
| DFDM19016-CK | Zhongkeyu 505 | 505 female parent × PH4CV | 29 | 33 | 87.9 |
| DFDM19017-5 | 505 female parent | 505 female parent × B6211 | 31 | 33 | 93.9 |
| DFDM19017-8 | 505 female parent | 505 female parent × B6232 | 26 | 33 | 78.8 |
| DFDM19017-6 | 505 female parent | 505 female parent × PH4CV | 28 | 33 | 84.8 |
| DFDM19017-15 | 505 female parent | 505 female parent × PH4CV | 29 | 33 | 87.9 |
| DFDM19018-CK | Xianyu 335 | PH6WC × PH4CV | 30 | 33 | 90.9 |
| DFDM19019-3 | PH6WC | PH6WC × B6275 | 27 | 33 | 81.8 |
| DFDM19019-4 | PH6WC | PH6WC × PH4CV | 29 | 33 | 87.9 |
| DFDM19019-7 | PH6WC | PH6WC × PH4CV | 23 | 33 | 69.7 |
| DFDM19019-8 | PH6WC | PH6WC × PH4CV | 29 | 33 | 87.9 |
| DFDM19019-9 | PH6WC | PH6WC × PH4CV | 27 | 33 | 81.8 |
| DFDM19019-11 | PH6WC | PH6WC × PH4CV | 28 | 33 | 84.8 |
| DFDM19019-12 | PH6WC | PH6WC × PH4CV | 20 | 33 | 60.6 |
| DFDM19019-13 | PH6WC | PH6WC × PH4CV | 32 | 33 | 97.0 |
| DFDM19019-14 | PH6WC | PH6WC × PH4CV | 29 | 33 | 87.9 |

TABLE 2
GEBV ranking of 60 cross combinations

| | Corresponding | GEBV |
|---|---|---|
| Cross combination | commercial hybrid | (g) |
| PH6WC × B6232 | | 213.4 |
| 505 female parent × B6232 | | 213.3 |
| A8111 × B6232 | | 212.9 |
| PH6WC × PH4CV | Xianyu 335 | 211.5 |
| 505 female parent × PH4CV | Zhongkeyu 505 | 211.4 |
| A8111 × PH4CV | | 211.0 |
| 303 female parent × B6232 | | 209.9 |
| A8001 × B6232 | | 209.3 |
| PH6WC × 605 male parent | | 208.8 |
| 505 female parent × 605 male parent | | 208.7 |
| PH6WC × B6275 | | 208.3 |
| A8111 × 605 male parent | | 208.2 |
| 505 female parent × B6275 | | 208.2 |
| 303 female parent × PH4CV | Yufeng 303 | 208.0 |
| A8111 × B6275 | | 207.7 |
| A8001 × PH4CV | | 207.3 |
| 303 female parent × 605 male parent | | 205.3 |
| 303 female parent × B6275 | | 204.8 |
| A8001 × 605 male parent | | 204.6 |
| A8001 × B6275 | | 204.1 |
| PH6WC × 618 male parent | | 203.7 |
| 505 female parent × 618 male parent | | 203.6 |
| 618 female parent × B6232 | | 203.2 |
| A8111 × 618 male parent | | 203.2 |
| PH6WC × B6211 | | 202.9 |
| 505 female parent × B6211 | | 202.8 |
| A8111 × B6211 | Lvyu 178 | 202.4 |
| 605 female parent × B6232 | | 202.0 |
| 618 female parent × PH4CV | | 201.2 |
| YD9953 female parent × B6232 | | 201.1 |
| DK517 female parent × B6232 | | 200.3 |
| 303 female parent × 618 male parent | | 200.2 |
| 605 female parent × PH4CV | | 200.1 |
| A8001 × 618 male parent | | 199.5 |
| 303 female parent × B6211 | | 199.4 |
| YD9953 female parent × PH4CV | | 199.2 |
| A8001 × B6211 | Luyan 188 | 198.7 |
| 618 female parent × 605 male parent | | 198.5 |
| DK517 female parent × PH4CV | | 198.3 |
| 618 female parent × B6275 | | 198.0 |
| 605 female parent × 605 male parent | Denghai 605 | 197.3 |
| 605 female parent × B6275 | | 196.8 |
| YD9953 female parent × 605 male parent | | 196.5 |
| YD9953 female parent × B6275 | | 196.0 |
| DK517 female parent × 605 male parent | | 195.6 |
| DK517 female parent × B6275 | | 195.1 |
| A7016 × B6232 | | 194.7 |
| 618 female parent × 618 male parent | Denghai 618 | 193.4 |
| A7016 × PH4CV | | 192.8 |
| 618 female parent × B6211 | | 192.6 |
| 605 female parent × 618 male parent | | 192.2 |
| 605 female parent × B6211 | | 191.4 |
| YD9953 female parent × 618 male parent | | 191.4 |
| YD9953 female parent × B6211 | | 190.6 |
| DK517 female parent × 618 male parent | | 190.5 |
| A7016 × 605 male parent | | 190.1 |
| DK517 female parent × B6211 | | 189.7 |
| A7016 × B6275 | Yayu No. 2 | 189.6 |
| A7016 × 618 male parent | | 185.0 |
| A7016 × B6211 | | 184.2 |

Finally, it should be noted that the above descriptions are only preferred examples of the present disclosure and are not intended to limit the present disclosure. Although the present disclosure is described in detail with reference to the above examples, a person skilled in the art can still make modifications to the technical solutions described in the foregoing examples, or make equivalent replacement to some technical features. Any modifications, equivalent substitutions, improvements, and the like made within the spirit and principles of the present disclosure should be included in the protection scope of the present disclosure.

What is claimed is:

1. A high-efficiency Zea mays L. breeding method based on an individual plant evaluation and a genome-wide selection (GWS), comprising the following steps:
S 1. in a first cropping season, pollinating a Zea mays L. female parent with multiple male parents;
S2. in a second cropping season, subjecting hybrid seeds to single-seed sowing, conducting an individual plant selection, and evaluating target traits;
S3. identifying a parent of a selected cross combination;
S4. subjecting a target trait of the selected cross combination to a genome-wide prediction;
S5. selecting an excellent cross combination according to a predicted target trait; and
S6. subjecting the excellent cross combination directly to a variety registration or to a further evaluation.

2. The high-efficiency Zea mays L. breeding method based on the individual plant evaluation and the GWS according to claim 1, wherein when the hybrid seeds are subjected to the single-seed sowing in S2, a female parent of the hybrid seeds subjected to the single-seed sowing is recorded.

3. The high-efficiency Zea mays L. breeding method based on the individual plant evaluation and the GWS according to claim 1, wherein during the single-seed sowing in S2, growth factors for individual plants are ensured to be consistent.

4. The high-efficiency Zea mays L. breeding method based on the individual plant evaluation and the GWS according to claim 1, wherein S3 specifically comprises the following steps:
S31. screening genomic DNAs (gDNAs) of all male and female parents in the first cropping season to obtain molecular markers used to identify parental genotypes;
S32. subjecting gDNA templates of all male and female parents in the first cropping season and the hybrid seeds to a polymerase chain reaction (PCR) amplification with the molecular markers, and recording genotypes;
S33. deriving all possible cross combination genotypes according to the genotypes of all male and female parents in the first cropping season; and
S34. comparing genotypes of the hybrid seeds with all possible cross combination genotypes derived, and recording a matching rate of identical genotype loci, wherein if the selected cross combination and a derived cross combination have a highest matching rate of identical genotype loci, a parent of the derived cross combination is a parent of the selected cross combination.

5. The high-efficiency Zea mays L. breeding method based on the individual plant evaluation and the GWS according to claim 1, wherein S4 specifically comprises the following steps:
S41. genotyping the parent of the selected cross combination identified in S3, and inferring a genotype of the selected cross combination according to a genotype of the parent;
S42. using target trait averages and genotypes of cross combinations evaluated in S2 to fit a genome-wide prediction model; and
S43. predicting target traits of all possible cross combinations according to the genome-wide prediction model.
